(19) United States
(12) Patent Application Publication
(10) Pub. No.: US 2025/0342920 A1
(43) Pub. Date: Nov. 6, 2025

(54) Standardization Of Reference Data For Electronic Health Records

(71) Applicant: Cerner Innovation, Inc., Kansas City, MO (US)

(72) Inventors: Vadim Khotilovich, Leawood, KS (US); Pragnya Ranjan Pradhan, Similipada (IN); Monica Gaur, Delhi (IN); Chetan KV, Bangalore (IN); Rupanjali Chaudhuri, Bangalore (IN); William Zimmerman, Kansas City, KS (US); Suman Pal, Bangalore (IN)

(73) Assignee: Cerner Innovation, Inc., Kansas City, MO (US)

(21) Appl. No.: 18/656,415

(22) Filed: May 6, 2024

(51) Int. Cl. G16H 10/60

(52) U.S. Cl. CPC G16H 10/60

(57) ABSTRACT

Techniques for generating recommendations of model domain entities from a model domain for mapping to comparison domain entities from a comparison domain are provided. A model domain includes a code set of standard references codes. A comparison domain includes a code set of reference codes that include non-standard reference codes. The reference codes represent clinical and non-clinical health concepts and are represented by one or more attributes. The system generates vector embeddings for entities of the comparison and model domains by applying a vector embedding function to the attributes fields of the comparison and model domain entities. The system compares the vector embeddings of the comparison domain entity to the vector embeddings of the model domain entity to compute similarity metrics for the entity pairs. The entity pairs are presented to a user based on the similarity metrics. A selected model domain entity is mapped to the comparison domain entity.

### TECHNICAL FIELD

The present disclosure relates to standardization of reference codes for clinical and non-clinical concepts. In particular, the present disclosure relates to creating a model domain of reference codes for semantic interoperability.

### BACKGROUND

Semantic interoperability enables healthcare systems to exchange data with unambiguous, shared meaning. Semantic interoperability is accomplished by linking each piece of data (a.k.a., entity or reference data) to a shared controlled vocabulary known as a terminology standard. Some examples of terminology standards include, but are not limited to: Systematized Nomenclature of Medicine Clinical Terms (SNOMED CT), Logical Observation Identifiers Names and Codes (LOINC), and International Classification of Diseases. Millions of entities are currently present in the medical ontology space with a rising addition of new entities. Multiple reference codes or entities may be used to identify the same concept. For example, system A may have an entity of ‘Male’ while system B may represent the same concept as ‘M’. As a result, healthcare data across various client domains is filled with ambiguous textual embeddings that may be present in the form of synonyms, acronyms, and abbreviations. This creates huge variance as the code values under various code sets are named differently though have semantic equivalence.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a system in accordance with one or more embodiments;

FIGS. 2A and 2B illustrate an example set of operations for mapping comparison domain entities to model domain entities in accordance with one or more embodiments;

FIG. 3 illustrates an example of data flow during an example set of operations for presenting a recommendation of a candidate model domain entity for entities of comparison domains;

FIG. 4 illustrates an interface for presenting recommendations of candidate model domain entities for mapping to comparison domain entities; and

FIG. 5 shows a block diagram that illustrates a computer system in accordance with one or more embodiments.

### DETAILED DESCRIPTION

In the following description, for the purposes of explanation, numerous specific details are set forth to provide a thorough understanding. One or more embodiments may be practiced without these specific details. Features described in one embodiment may be combined with features described in a different embodiment. In some examples, well-known structures and devices are described with reference to a block diagram form to avoid unnecessarily obscuring the present disclosure.
1. GENERAL OVERVIEW
2. STANDARDIZED REFERENCE CODE MAPPING SYSTEM
3. RECOMMENDING A CANDIDATE MODEL DOMAIN ENTITY FOR MAPPING TO A COMPARISON DOMAIN ENTITY
4. EXAMPLE MAPPING OPERATIONS
5. RECOMMENDATION INTERFACE
6. PRACTICAL APPLICATIONS, ADVANTAGES & IMPROVEMENTS
7. HARDWARE OVERVIEW
8. MISCELLANEOUS; EXTENSIONS

### 1. General Overview

One or more embodiments generate recommendations for mapping (a) model domain entities from a model domain to (b) comparison domain entities from a comparison domain. A model domain, as referred to herein, includes a code set of standard reference codes or entities. A comparison domain, as referred to herein, includes a code set of reference codes or entities that include non-standard reference codes or entities. The entities of the comparison domain and the model domain represent clinical and non-clinical health concepts and are represented by one or more attributes.

Initially, the system generates vector embeddings for entities of the comparison domain by applying a vector embedding function to the textual attributes fields of the comparison domain entities. Similarly, the system generates vector embeddings for the entities of the model domain by applying the same vector embedding function to the textual attribute fields of the model domain entities. Entity pairings are created for the entities of the model domain and the comparison domain. The system compares the vector embedding of the comparison domain entity to the vector embedding of the model domain entity for the entity pairing. Based on the similarity metrics for the entity pairings, the system sorts the entity pairings.

In one or more embodiments, the entity pairings that have a similarity metric that exceed a threshold are presented to a user as candidate entity pairings. The entity pairings may be presented as likely matches or possible matches. The system refrains from presenting entity pairings that have a similarity metric that are below the threshold. A comparison domain entity for a health concept that is not similar to a health concept of a model domain entity is presented as a “comparison-only” entity. Similarly, a model domain entity for a health concept that is not similar to a health concept of a comparison domain entity is presented to the user as a “standard-only” entity.

In one or more embodiments, the system receives user input indicating that the health concept of the comparison domain entity of the selected entity pairing and the health concept of the model domain entity of the selected entity pairing are a match. Responsive to receiving the user input, the model domain is updated to reflect the match between the health concept of the model domain entity and the health concept of the comparison domain entity. The system then uses the updated model domain to facilitate exchange of health code data between a first healthcare system and a second healthcare system.

One or more embodiments described in this Specification and/or recited in the claims may not be included in this General Overview section.

### 2. Standardized Reference Code Mapping System

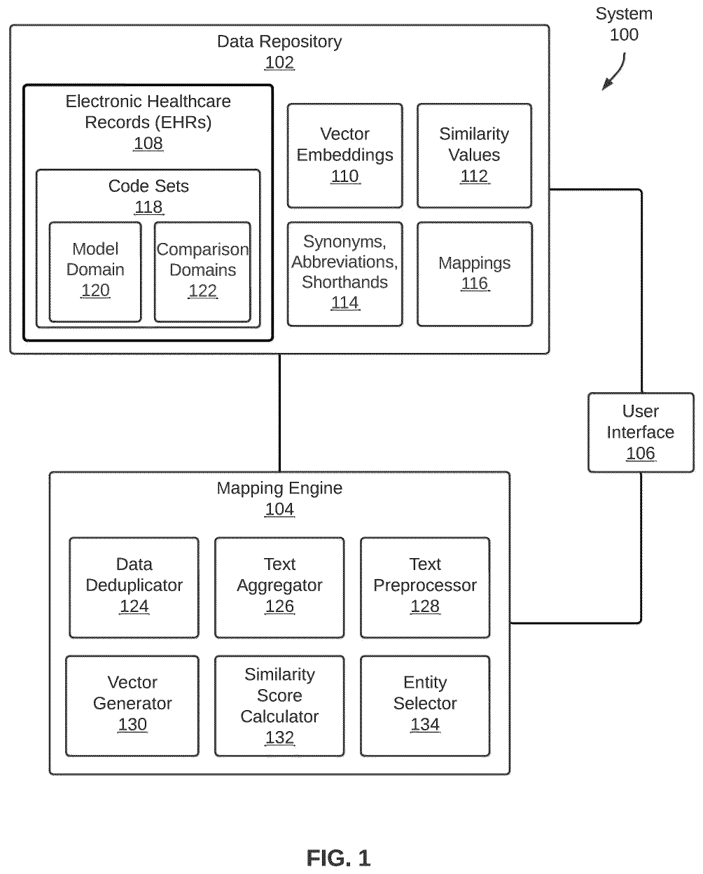

FIG. 1 illustrates a system 100 in accordance with one or more embodiments. As illustrated in FIG. 1, system 100 includes a data repository 102, a mapping or recommendation engine 104, and a user interface 106. In one or more embodiments, the system 100 may include more or fewer components than the components illustrated in FIG. 1. The components illustrated in FIG. 1 may be local to or remote from each other. The components illustrated in FIG. 1 may be implemented in software and/or hardware. Each component may be distributed over multiple applications and/or machines. Multiple components may be combined into one application and/or machine. Operations described with respect to one component may instead be performed by another component.

In one or more embodiments, a data repository 102 is any type of storage unit and/or device (e.g., a file system, database, collection of tables, or any other storage mechanism) for storing data. Further, a data repository 102 may include multiple different storage units and/or devices. The multiple different storage units and/or devices may or may not be of the same type or located at the same physical site. Further, a data repository 102 may be implemented or executed on the same computing system as the mapping engine 104 and the user interface 106. Alternatively, or additionally, a data repository 102 may be implemented or executed on a computing system separate from the mapping engine 104 and the user interface 106. The data repository 102 may be communicatively coupled to the mapping engine 104 and the user interface 106 via a direct connection or via a network.

Information describing operations for recommending model domain entities corresponding to comparison domain entities may be implemented across any components within the system 100. However, this information is illustrated within the data repository 102 for purposes of clarity and explanation.

In embodiments, the data repository 102 is populated with information from a variety of sources and/or systems. The data repository 102 may include electronic healthcare records (EHRs) 108. The EHRs 108 are populated with reference codes or entities. The entities may be organized into code sets 118. The code sets 118 may include entities from a model domain 120 and one or more comparison domains 122. The data repository 102 may further include vector embeddings 110, similarity values 112, synonyms, abbreviations, and shorthands 114, and mappings 116. Any of this information may be stored in a structured format (e.g., a table).

In one or more embodiments, the EHRs are digital versions of patients' paper charts that include at least portions of the patients' medical histories. The EHRs 108 may be from the same or different systems and/or providers. Some examples of EHR providers include, but are not limited to, Cerner Millenium and Epic. The EHRs 108 are populated with reference codes or entities that represent clinical and non-clinical concepts. The reference codes may be organized into code sets 118. Different EHRs may have different code sets for organizing the entities and/or different entities for identifying the same clinical and non-clinical concepts.

In one or more embodiments, a code set refers to a standardized system of codes used to represent various medical concepts, procedures, diagnoses, medications, and other healthcare-related information. The code sets 118 are used for a variety of purposes, including billing, reimbursement, clinical documentation, research, and data analysis. The code sets 118 ensure consistency, accuracy, and interoperability of healthcare information across different systems and organizations.

In one or more embodiments, the code sets 118 are organized in a structured manner to represent various concepts, items, or processes within a particular domain. Many code sets are organized hierarchically, with codes grouped into categories, subcategories, and levels of detail. A code set for an EHR provider may include, for example, “Route,” “Body Site,” “Order Type,” and/or “Sex.” This hierarchical structure allows for easy navigation and classification of codes. In the International Classification of Diseases (ICD), codes are organized into chapters, sections, and subcategories based on the type of disease or condition. The code sets 118 may use numeric or alphanumeric codes to represent different concepts or items. Numeric codes are often sequential and may be organized based on specific criteria, such as in the order that the codes were introduced. Alphanumeric codes may contain letters and numbers and may follow specific patterns or formats. Code sets 118 are often organized using categorization schemes that group related codes together based on common characteristics or attributes. These categorization schemes may be defined by standard-setting organizations or regulatory bodies. For example, in the Healthcare Common Procedure Coding System (HCPCS), codes are categorized into different levels (Level I, Level II, Level III) based on the type of service or item being coded. Code sets 118 may include cross-references or mappings to related codes in other code sets. This allows users to easily find equivalent codes or codes that are related to a specific concept across different code sets. Cross-references help ensure consistency and interoperability between different systems and code sets. Code sets 118 are often organized according to standardized formats and terminologies defined by standard-setting organizations or regulatory bodies. These standards specify the structure, syntax, and semantics of codes, as well as rules for their use and interpretation. Adherence to standard formats and terminologies helps ensure consistency, accuracy, and interoperability of information.

In one or more embodiments, the model domain 120 is an exhaustive set of reference codes or entities for describing clinical and non-clinical concepts. The code sets of the model domain 120 are standardized groupings of the reference codes or entities from a specific domain or field. The model domain 120 may be particular to an EHR provider, an organization, or an industry.

In one or more embodiments, the model domain 120 includes mappings for industry standard codes, proprietary codes, and organization specific codes. Industry standard codes are sets of reference codes commonly used in the healthcare industry. Example industry standard codes include Systematized Nomenclature of Medicine-Clinical Terms (SNOMED CT), International Classification of Diseases (ICD), Logical Observation Identifiers Names and Codes (LOINC), Current Procedural Terminology (CPT), Unified Code for Units of Measure (UCUM), Healthcare Common Procedure Coding System (HCPCS), and National Drug Code (NDC). SNOMED CT is a comprehensive clinical terminology system used to represent and encode clinical information in electronic health records (EHRs) and other healthcare systems. Logical Observation Identifiers Names and Codes (LOINC) is a universal standard for identifying health measurements, observations, and clinical documents. ICD is used to classify and code diagnoses, symptoms, and procedures for medical billing and statistical purposes. CPT codes are developed by the American Medical Association and are used to describe medical procedures and services provided by healthcare professionals for billing and reimbursement purposes. UCUM a standardized system for representing units of measurement in healthcare and other domains. HCPCS Complements CPT codes and include additional codes for services, supplies, and equipment not covered by CPT codes. NDC is a unique 10-digit code used to identify specific prescription and over-the-counter drugs in the United States. Organization specific codes may include Cerner Knowledge Index (CKI) and Concept CKI (CCKI).

In an embodiment, entities within the model domain 120 are identified by attributes. A model domain having multiple domains may be identified using “Client-Domain” label where client refers to the client's name and domain is the name of the domain. A model domain may be divided into “Code Sets.” A code set represents an entity type category that consists of entities belonging to a particular type. For example, “cs_6006” is a code set for “Order Type,” “cs 1028” is a code set for “Body Site,” and “cs_1306” is a code set for Specimen. Attributes for entities within a “Code Set” of the model domain may include “Code Value,” “Display,” “Description,” “Definition.” “Code Value” is an identifier assigned to an entity. “Display” is the display name of an entity. “Description” is a description of an entity. “Definition” is a definition of an entity.

In one or more embodiments, the entities within a model domain may include alternative attributes. Alternative attributes for an entity may include “CKI,” “CKI Display,” “Concept CKI,” “Concept CKI Display,” “Standard Code System,” “Industry Standard Code,” “Standard Code Name.” “CKI” refers to a Cerner Knowledge Index-Cerner specific codes created to represent a certain concept. “CKI Display” is the display name of the CKI. “Concept CKI (cCKI)” captures more granular concepts that may not be covered by CKI. “Concept CKI Display” is the display name of the cCKI. “Standard Code System” refers to an industry-standard code system like SNOMED CT, LOINC, UCUM. “Standard Code Name” is the standard name of an entity as per the Standard Code System.

In one or more embodiments, the comparison or local domain is a set of reference codes or entities for describing clinical and non-clinical concepts. The code sets of the comparison domain include standardized codes and non-standardized codes for entities. The code sets of the comparison domain may be based on an initial code set of an EHR provider that has been modified for local practice. More specifically, when needs of local practice are favored over uniformity of content, clients may create or customize their own reference codes or entities. Entities of the comparison domain may include different or more specific entities from entities of the model domain. For example, an entity in a model domain may be identified as “lung” and a similar entity in a comparison domain may be identified as “lung—right.” The comparison domain main further include an entity identified as “lung—left.” Another comparison domain may include an entity identified as “lung—both” or “lung—right & left.” Still another comparison domain may divide the right and left lungs into sections, with entities provided for the sections, e.g., “lung—upper lobe,” “lung—middle lobe” and “lung—lower lobe,” and/or “lung—upper division” and “lung—lower division.”

In one or more embodiments, the vector embeddings 110 in the data repository 102 include text that has been converted to a numeric format. The vector embeddings 110 are representations of individual words for text analysis, typically in the form of a real-valued vector. The vector embeddings 110 may represent individual text items or may represent an aggregation of text items. As will be described in further detail below with respect to mapping engine 104, the vector embeddings 110 may be formed using various word embedding techniques. The vector embeddings 110 represent entities in the code sets of the model domain and the comparison domains. The text represented by the vector embeddings 110 includes the entries for the attribute fields of the entities, including, for example, “Description,” “Display,” and “Definition.”

In some embodiments, the similarity values or metrics 112 in the data repository 102 provide an indication of the similarity between the vector embeddings 110 for entities of the model domain 120 and entities of the comparison domains 122. The higher the similarity values 112 (for example, the closer to 1.0, depending on the scale), the greater a semantic match between the vector embeddings 110 of a model domain entity and a comparison domain entity. The similarity values 112 may be assigned a ranking category. For example, a similarity value less than 0.90 may be categorized as “low”; a similarity value equal to or greater than 0.90 and less than 0.98 may be categorized as “medium”; and a similarity value greater than or equal to 0.98 may be categorized as “high.” Alternatively, the similarity value may be used to categorize entity pairs as a “likely” match or a “possible” match. A model domain entity that does not appear in a comparison domain may be categorized as “standard-only” and a comparison domain entity that does not appear in the model domain may be categorized as “comparison-only.” The similarity values 112 may be weighted to reflect the relevance of the type of data used to calculate the vector embeddings 110. For example, attributes with a high relevance to determining an appropriate mapping of entities may receive a weight of 0.55, while data with less relevance to the mapping may receive a weight of 0.45.

In some embodiments, the synonyms, abbreviations, and shorthands 114 are included in a table that provides synonyms, abbreviations, and/or shorthands that may or may not be specific to a consumer and corresponding expansions for the respective synonym, abbreviation or shorthand. For example, “SBP” may correspond to “systolic blood pressure”; “LMP” may correspond to “last menstrual period”; “I:E” may correspond to “inspiratory to expiratory ratio”; and “GAD7” may correspond to “general anxiety disorder.”

In embodiments, mappings 116 include mappings of entities in the comparison domain that correspond to entities in the model domain. Mappings 116 may also include mappings of entities in the model domain and/or comparison domains to entities in the industry standard domains, e.g., SNOMED CT, UCUM, LOINC or organization specific domains, e.g., CKI, cCKI.

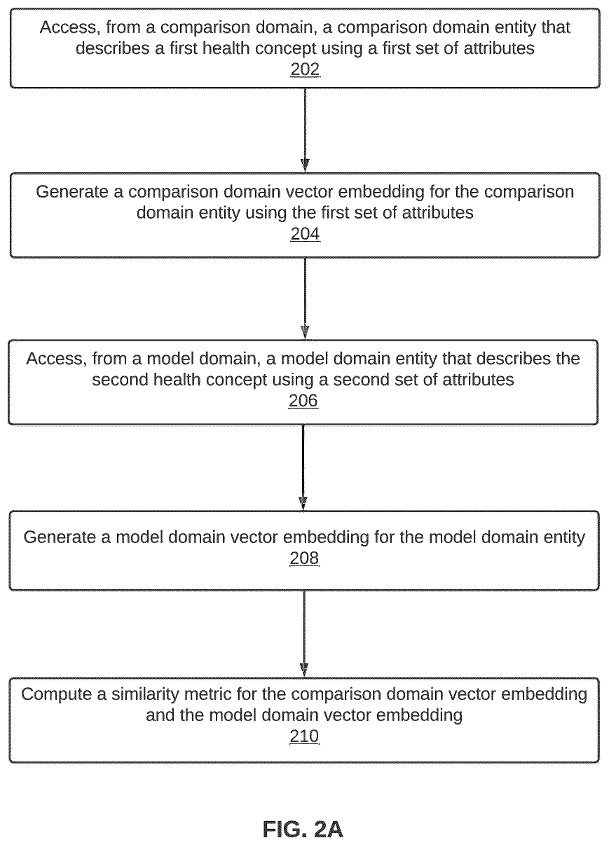

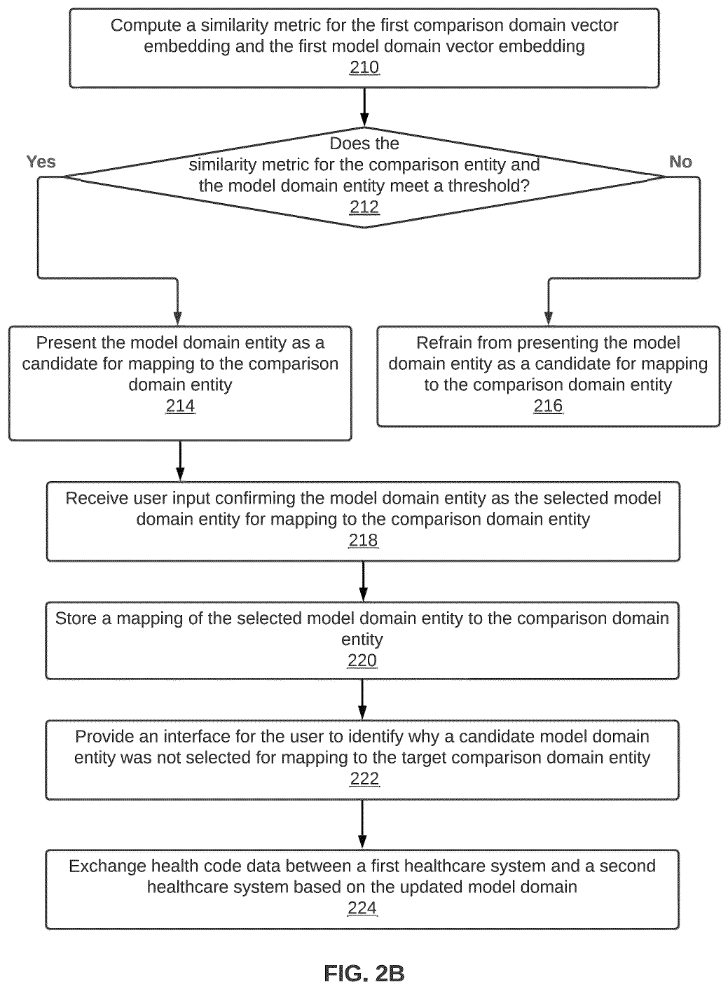

In embodiments, the mapping engine 104 of the system 100 is hardware and/or software configured to recommend entities in the model domain that may correspond to entities in the comparison domain. Examples of operations for providing recommendations of candidate model domain entities for comparison domain entities are described below with references to FIGS. 2A and 2B. The mapping engine 104 may include a data deduplicator 124, a text aggregator 126, a text preprocessor 128, a vector generator 130, a similarity score calculator 132, and an entity selector 134.

In one or more embodiments, the data deduplicator 124 is a component of the mapping engine 104 that removes entities from the comparison domains that have the same attributes. For example, when “Display,” “Description,” and “Definition” are attributes used to compare entities of a model domain to entities of comparison domains, an entity of a second comparison domain that includes the same attribute fields or entries as an entity of a first comparison domain is considered a duplicate. The duplicate is removed, and a single cross domain ID is assigned to the entity of both the first comparison domain and second comparison domain. Conversely, an entity of the second comparison domain that includes one or more different entries for attributes from an entity of the second comparison is considered a unique entity. A first cross domain ID is assigned to the entity of the first comparison domain and a second cross domain ID is assigned to the entity of the second comparison domain. Data deduplication is performed to optimize pair generation as the amount of data for various code sets may be extensive.

In one or more embodiments, the text aggregator 126 aggregates text from the attribute fields of the entities of the model domain 120 and the attribute fields of the entities of the comparison domains 122. The text aggregator 126 may aggregate text prior to preprocessing of the text by the text preprocessor 128 or after preprocessing of the text for the attributes.

In some embodiments, the text of the attribute fields of the entities for the model and comparison domains is processed by the text preprocessor 128 prior to applying the vector generator 130 to the aggregated text to generate vector embeddings 110. The text preprocessor 128 may perform functions such as converting the text into lowercase, removing white spaces, prefix removal, punctuation removal, and/or retaining numeric tokens. Text is converted to lowercase to provide uniformity to the text. Prefix removal includes removing prefixes such as “z,” “zz,” “zzz.” Punctuation removal is performed to remove any non-alphanumeric characters. In prior art mapping engines, numeric tokens are typically removed during text preprocessing. Removal of numeric tokens may eliminate a distinguishing feature of a concept. For example, “Right Ear 500 Hz POC” and “Right Ear 1000 Hz POC” are differentiated using a numeric token. By retaining numeric tokens, mismatches are more readily avoided.

In embodiments, text preprocessing may further include handling special characters, removing unwanted text, and custom preprocessing. Handling special characters includes addressing symbols and special characters. For example, text line “D-Dimer” requires special attention. Replacing the “-” with a blank space creates two different tokens, namely “D” and “Dimer.” As such, using traditional text preprocessing, the entire context of “D-Dimer” is lost. By addressing special characters, the context of the terms is maintained. Custom preprocessing includes attending to consumer specific text such as synonyms, abbreviations, and shorthands. The custom preprocessing may consult the synonyms, abbreviations, and shorthands 114 stored in the data repository 102 to provide expansions for various consumer specific synonyms, abbreviations, and shorthands.

In some embodiments, the vector generator 130 includes software and/or hardware for performing one or more vector embedding functions. Vector embedding functions are mathematical functions that map objects, such as words, sentences, or other data points, into vector representations in a multi-dimensional space. These vector representations are used to capture the semantic or contextual meaning of the objects in a numerical format that can be easily processed by machine learning algorithms.

In some embodiments, the vector embedding functions are word embedding techniques. Word embedding techniques use natural language processing (NLP) and machine learning to represent words as dense vectors of real numbers. Word embedding techniques aim to capture the semantic and syntactic meaning of words as well as their relationships with other words in a language. Word embedding techniques include Term Frequency-Inverse Document Frequency (TF-IDF), Word2Vec, Global Vectors (GLOVE), Large Language Models (LLM), BioWordVec fastText, and Bidirectional Encoder Representation (BERT).

Each of these word embedding techniques includes salient features. The TF-IDF model is designed to give more weight to the words that are very specific to certain documents but give less weight to the words that are more general and occur across most documents. The Word2Vec model represents words in the form of dense vectors by capturing syntactic (grammar) and semantic (meaning) relationships. Given a large enough dataset, the Word2vec model provides strong estimates about a meaning of a word based on the frequency of occurrence of the word in the text. The GLOVE model is an unsupervised learning model that can be used to obtain dense word vectors like the Word2Vec model. The GLOVE model first creates a large word-context, co-occurrence matrix consisting of pairs (word, context). Each element in this matrix represents how often a word or a sequence of words occurs within the context and then applies matrix factorization to approximate this matrix. The BioWordVec fastText model is 200-dimensional word embeddings trained on PubMed and MIMIC-III data and is the extension of the original BioWordVec that provides fastText word embeddings trained using PubMed and MeSH. A subword embedding model used by the Bio WordVec fastText model better handles out of vocabulary tokens and improves the quality of the word embeddings. BERT uses encoder-only transformer architecture that learns the contextual relations between words (or subwords) in textual data and converts text into embeddings. BERT is trained on an unsupervised task of ‘Mask Language Model (MLM)’ using text corpora from BooksCorpus and English Wikipedia

In one or more embodiments, the word embedding techniques include Self-Alignment Pretraining for Biomedical Entity Representations (SAPBERT). The SAPBERT is a pre-trained BERT model that is trained on Medical Entity Linking (MEL) tasks. MEL maps various entities to unified concepts in the medical knowledge graph. Word representation learning faces a significant challenge due to the existence of heterogeneous names. For example, in healthcare, terms like ‘nostril’ and ‘nare’ are used interchangeably but yield considerably different embedding representations when generated by models not specifically trained for MEL. SAPBERT works on self-alignment of biomedical entity representation such that the semantically similar entities belonging to the same concept are brought closer in the embedding space, thus forming compact clusters. SAPBERT leverages UMLS, the largest collection of biomedical concepts and synonyms and collates the synonyms from various controlled vocabularies, e.g., SNOMED CT, MeSH, Gene Ontology, RxNorm, and OMIM. SAPBERT performs better compared to other variants of BERT like Bio-BERT, Clinical-BERT with respect to the MEL challenges. The SAPBERT model can accurately capture fine-grained semantic relationships and heterogeneous naming in the biomedical domain compared to other variants of BERT. The ability of SAPBERT to handle out-of-vocabulary terms, misspelled words, and rare medical terms provides a significant advantage over other models.

In embodiments, the similarity score calculator 132 calculates a similarity between vector embeddings for entities of the model domain and vector embeddings for entities of the comparison domains. Similarity matching or similarity retrieval can be used to find items, e.g., model domain entities, that are similar to a given query item, e.g., comparison domain entity. Similarity matching measures the similarity between an entity of a comparison domain and an entity of the model domain, based on certain features or characteristics, i.e., attributes, and then ranks the entity pairs by their similarity. To measure similarity, a distance measure or similarity metric is chosen. Common distance measures include Euclidean distance, cosine similarity, and Jaccard similarity. When dealing with large data sets, an index may be created to speed up the search process. An index is a data structure that organizes the data in a way that allows for efficient retrieval of similar items.

In one or more embodiments, the similarity score calculator 132 includes the Facebook AI Similarity Search (FAISS). FAISS is an open-source library developed by Facebook for efficient similarity search and clustering of high-dimensional vectors. FAISS is optimized for both CPU and GPU architectures, enabling fast and scalable similarity search operations on large datasets. FAISS supports a range of similarity metrics, including Euclidean distance, cosine similarity, inner product, and L2 distance. FAISS offers various indexing methods, including the flat index, inverted file (IVF), Hierarchical Navigable Small World (HNSW), and product quantization. Flat index uses an index built from data points without any hierarchical structure. When a search operation is performed, the distance between the query vector and all the other vectors utilized to build the index is computed and the top-n closest vectors are returned. When using IVF, a dataset is divided into clusters using a clustering algorithm (e.g., k-means). Each cluster is associated with a unique identifier. For each cluster, an inverted list is created. An inverted list is a data structure that associates a cluster identifier with the list of vectors that belong to that cluster. During indexing, each data vector is assigned to the nearest cluster centroid. This assignment is used to determine the inverted list to update with the vector's information. When performing a similarity search, the query vector is quantized to the nearest cluster centroid. FAISS then searches the inverted list associated with that cluster for potential nearest neighbors. HNSW is an algorithm for efficient similarity search in high-dimensional spaces. These indexing techniques help speed up nearest-neighbor searches in high-dimensional spaces.

In an embodiment, FAISS is combined with HNSW as the indexing approach. FAISS can be integrated with popular machine learning libraries and frameworks, such as PyTorch and TensorFlow, making it easier to incorporate similarity searches into machine learning pipelines. This may lead to significant improvements in the speed and scalability of the similarity search operations. As an open-source library, FAISS is available for developers and researchers to use, modify, and contribute to the development FAISS.

In one or more embodiments, recommendations for model domain/comparison domain entity pairs are provided by the entity selector 134. The entity selector 134 presents model domain/comparison domain entity pairs to the user interface 106 based on the similarity values 112 provided by the similarity score calculator 132. The entity selector 134 may present an “N” number of candidate model domains for mapping to a comparison domain and/or model domain/comparison domain entity pair ranked by the similarity values between the vector embeddings of the model domain entity and the vector embedding of the comparison domain entity. Alternatively, the entity selector 134 may present every model domain/comparison domain entity pairing having a similarity measure above a threshold. Depending on the similarity values, a candidate model domain and/or an entity pair may be identified as “likely” or “possible.” Selection of a candidate model domain and/or an entity pair updates the model domain to reflect the match between the model domain entity and the comparison domain entity.

In one or more embodiments, the entity selector 134 presents a model domain entity that is not paired with a comparison domain entity as a “standard-only” entity. Similarly, the entity selector 134 may present comparison domain entities that are not paired with a model domain entity as “comparison-only.” The user may select a comparison domain entity that is identified as “comparison-only” for adding to the model domain. The addition of a “comparison-only” entity from the comparison domain to the model domain creates a more exhaustive set of entities for future mapping.

In an embodiment, the mapping engine 104 is implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a client device.

In one or more embodiments, user interface 106 refers to hardware and/or software configured to facilitate communications between a user and the mapping engine 104. User interface 106 renders user interface elements and receives input via user interface elements. Examples of interfaces include a graphical user interface (GUI), a command line interface (CLI), a haptic interface, and a voice command interface. Examples of user interface elements include checkboxes, radio buttons, dropdown lists, list boxes, buttons, toggles, text fields, date and time selectors, command lines, sliders, pages, and forms.

In an embodiment, different components of user interface 106 are specified in different languages. The behavior of user interface elements is specified in a dynamic programming language, such as JavaScript. The content of user interface elements is specified in a markup language, such as hypertext markup language (HTML) or XML User Interface Language (XUL). The layout of user interface elements is specified in a style sheet language, such as Cascading Style Sheets (CSS). Alternatively, user interface 106 is specified in one or more other languages, such as Java, C, or C++.

### 3. Recommending Model Domain Entities for Mapping to Comparison Domain Entities

FIGS. 2A and 2B illustrate an example set of operations for recommending model domain entities for mapping to comparison domain entities in accordance with one or more embodiments. One or more operations illustrated in FIGS. 2A and 2B may be modified, rearranged, or omitted all together. Accordingly, the particular sequence of operations illustrated in FIGS. 2A and 2B should not be construed as limiting the scope of one or more embodiments.

One or more embodiments access, from a comparison domain, a comparison domain entity that describes a first health concept using a first set of attributes (Operation 202). The comparison domain is a set of one or more reference code sets used by a client, e.g., a hospital, of an electronic healthcare record (EHR) provider to describe clinical and not clinical concepts. The code sets may include standard entities and entities that are customized to the needs of local practice. The entities in the comparison domain represent unique health concepts and are identified with a plurality of attributes. The attributes for the comparison domain entity may include “Description,” “Display” and “Definition.” The comparison domain entity may include an entry for some or all attribute fields.

One or more embodiments generate a comparison domain vector embedding for the comparison domain entity using the first set of attributes (Operation 204). A vector embedding function generates a vector embedding for the comparison domain entity. The vector embeddings are numerical representations of aggregated text from the attribute fields of the comparison domain entity. The vector embedding function may include Self-Alignment Pretraining for Biomedical Entity Representations (SAPBERT). Prior to generating the vector embedding, the text from the attribute fields of the comparison domain entity may be preprocessed. Preprocessing the text provides uniformity to the text. The text may also be aggregated prior to generating the vector embedding.

Along with generating the vector embedding for the comparison domain entity, the system may generate vector embeddings for other entities in the comparison domain. Similarly, the system may generate vector embedding for entities in other comparison domains. In this manner, one or more additional comparison domains may be processed at the same time as the comparison domain. The system may compare the attribute fields of entities across the additional comparison domains and remove entities with attribute fields that are the same as the attribute fields of the entities of the comparison domain, i.e., deduplication.

One or more embodiments access, from a model domain, a model domain entity that describes the second health concept using a second set of attributes (Operation 206). The model domain is a set of one or more reference code sets maintained by the EHR provider to describe clinical and non-clinical concepts. The model domain is intended to be an exhaustive set of entities for use by clients of the EHR provider. The code sets of the model domain may be the same or different from the code sets of the comparison domain. Differences in code sets between the comparison domain and the model domain may result from the comparison domain being developed by a different EHR provider or from customizations made by the client of the EHR provider. The model domain entity is identified using a plurality of attributes. The attributes for the model domain entity may be the same or different from the attributes of the comparison domain entity.

One or more embodiments generate a model domain vector embedding for the model domain entity (Operation 208). The same vector embedding function used to generate the vector embedding for the comparison domain entity is used to generate a vector embedding for the model domain entity. Prior to generating the vector embedding, the text for the attribute fields of the model domain entity may be preprocessed. The text may also be aggregated prior to generating the vector embedding. Along with generating the vector embedding for the model domain entity, vector embeddings may be generated for other entities in the model domain.

One or more embodiments compute a similarity metric for the comparison domain vector embedding and the model domain vector embedding (Operation 210). The similarity metric or similarity value is a semantic similarity between the comparison domain vector embedding for the comparison domain entity and the model domain vector embeddings for the model domain vector embedding. The similarity metric may be calculated using Facebook AI Similarity Search (FAISS). FAISS may be combined with Hierarchical Navigable Small World (HNSW) as the indexing approach.

In one or more embodiments, the system computes similarity metrics for the other entity pairs from the comparison domain and model domain. Similarity metrics may also be computed for the entity pairs of the entities of the model domain and the entities of the one or more additional comparison domains.

One or more embodiments determine if the similarity metric for the comparison entity and the model domain entity meets a threshold (Operation 212). The closer to 1.0 the similarity metric, the greater the likelihood of the comparison domain entity and the model domain entity being a match. A threshold similarity metric may include a similarity metric above a predetermined measure, e.g., above 0.90. The threshold may include a first threshold having a first similarity metric and a second threshold having a second, higher threshold.

In embodiments, the system also determines if the similarity metrics computed for the entity pairs of the other entities in the comparison domain and the model domain entity and/or the other entities of the model domain meet the threshold. The system may also determine if the similarity metrics for the other entity pairs of the one or more additional comparison domains and the entities of the model domain meet the threshold.

One or more embodiments present the model domain entity as a candidate for mapping to the comparison domain entity (Operation 214). When the similarity metric for the comparison domain entity and the model domain entity meets a first threshold, the model domain entity may be presented as a “possible” match for mapping to the comparison domain entity. When the similarity metric for the comparison domain entity and the model domain entity meets a second, higher threshold, the model domain entity may be presented as a “likely” match for mapping to the comparison domain entity.

One or more embodiments refrain from presenting the model domain entity as a candidate for mapping to the comparison domain entity (Operation 216). When the similarity metric for the comparison domain entity and the model domain entity is below the threshold, the system refrains from presenting the model domain entity as a match for mapping to the comparison domain entity. When the system fails to match the model domain entity with a comparison domain entity, the model domain entity may be presented as a “standard-only” entity. Similarly, when the system fails to match the comparison domain entity with a model domain entity, the comparison domain entity may be presented as a “comparison-only” entity. An entity identified as “standard-only” is not present in the comparison domains and an entity identified as “comparison-only” is not present in the model domain.

One or more embodiments receive user input confirming the model domain entity as the selected model domain entity for mapping to the comparison domain entity (Operation 218). The user input may include selecting an icon or text representing the desired candidate model domain entity for matching with the comparison domain or the comparison domain entity for matching with the model domain. Alternatively, the user input may include selecting an icon or text representing the model domain/comparison domain entity pair.

One or more embodiments store a mapping of the selected model domain entity to the comparison domain entity (Operation 220). Receipt of user confirmation of an entity pair match causes the system to store the mapping of the comparison domain entity to the selected model domain entity.

One or more embodiments provide an interface for the user to identify why a candidate model domain entity was not selected for mapping to the target comparison domain entity (Operation 222). To assist in understanding why certain entity pairings were not selected, the interface for selecting the entity pair may include a text box, buttons or other features for a user to provide feedback regarding the selected entity pairs. The feedback may be used to retrain or adjust the models used for vector generating and/or calculating similarity metrics.

One or more embodiments exchange health code data between a first healthcare system and a second healthcare system based on the updated model domain (Operation 224). The first healthcare system uses a first comparison domain for identifying entities and the second healthcare system uses a second comparison domain for identifying entities. The first and second comparison domains may be from the same or different EHR providers. The additional mapping provided by the selection of the model domain entity for the comparison domain entity allows for improved exchange of health code data from the first comparison domain to the second comparison domain using the model domain. More particularly, the entities of the first comparison domain are mapped to the entities of the model domain. Similarly, the entities of the second comparison domain are mapped to the entities of the model domain. Using the model domain entities as common mapping entities, the system automatically populates the entities from the first healthcare system into the correct locations in the second healthcare system.

### 4. Example Mapping Operations

A detailed example is described below for purposes of clarity. Components and/or operations described below should be understood as one specific example which may not be applicable to certain embodiments. Accordingly, components and/or operations described below should not be construed as limiting the scope of any of the claims.

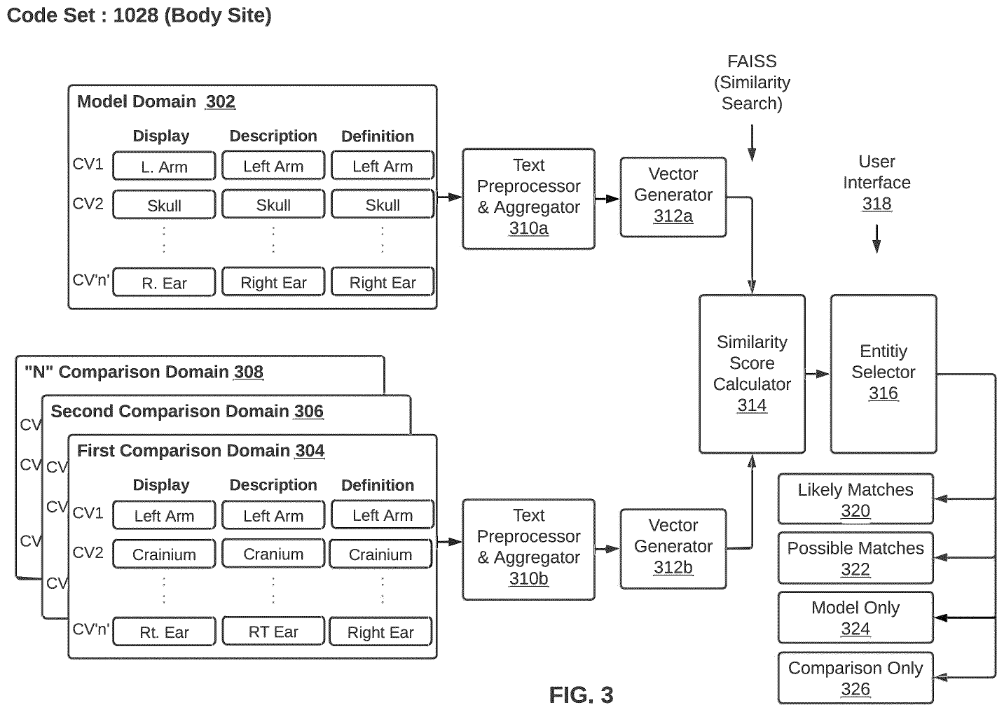

FIG. 3 illustrates operations for providing recommendations for mapping of model domain entities to entities of comparison domains. The model domain and the comparison domains are from a code set identified as Code Set: 1028 (Body Site). Initially, the attribute fields for the entities in the model domain 302 and the first comparison domains 304, second comparison domain 306, and “N” comparison domain 308 are obtained. Although shown including multiple comparison domains, the operations are equally applicable to a single comparison domain. As shown, the attributes for the model domain and the comparison domains are the same. When the comparison domains are from a different EHR provider from the model domain, the attributes for the comparison domain may be different from the attributes for the model domain. The attributes include “Display,” “Description,” and “Definition.” Although shown including an entry in each attribute field for the entities, not all attribute fields of all entities may include an entry.

A text preprocessor and aggregator 310a is applied to the text in the attribute fields for each of the entities or code values (CV), in the model domain 302. Similarly, a text preprocessor and aggregator 310b is applied to the text in the attribute fields for each of the entities or code values in the comparison domain 304, 306, 308.

A vector generator 312a is then applied to the aggregated and preprocessed text of the entities of the model domain 302 to generate vector embeddings for the entities of the model domain 302. Similarly, a vector generator 312b is applied to the aggregated and preprocessed text of the entities of the comparison domains 304, 306, 308 to generate vector embeddings for the entities in the comparison domains 304, 306, 308.

A similarity score calculator 314 takes the vector embeddings for the entities of the model domain and comparison domains and computes similarity metrics for the entity pairs.

An entity selector 316 presents the top “K” entity pairs to a user on a user interface 318. The top “K” entity pairs are ranked based on the similarity scores computed by the similarity score calculator 314.

The entity pairs determined to have the highest similarity score, i.e., above a first threshold, are identified as Likely Matches 320. The entity pairs determined to be above a second, lower threshold are identified as Possible Matches 322. Model domain entities that do not appear in any of the comparison domains, as determined by their similarity scores, are identified as Model Only 324, and comparison domain entities that do not appear in the model domain, as determined by their similarity scores, are identified as Comparison Only 326.

### 5. Recommendation Interface

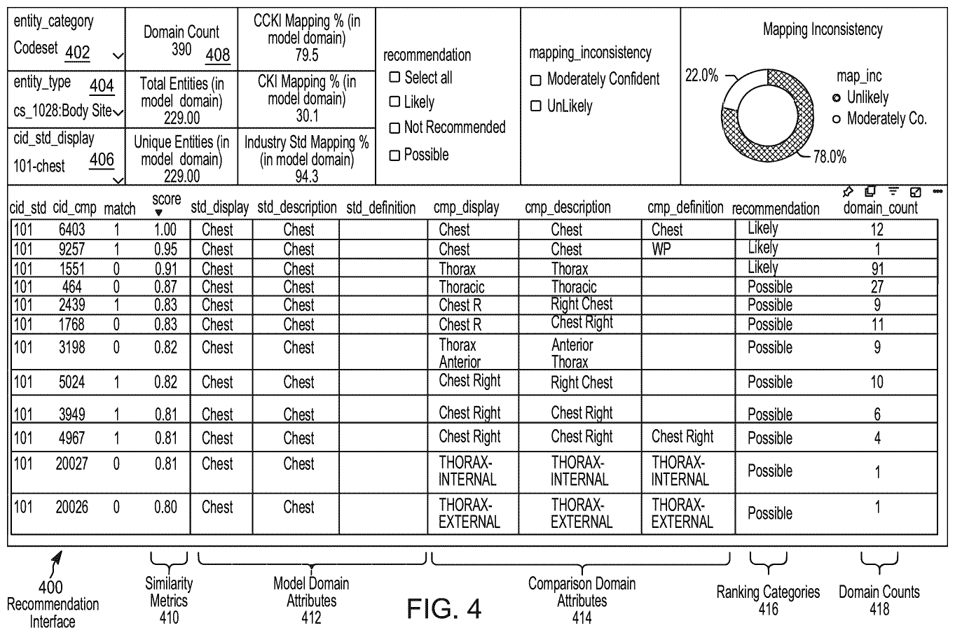

FIG. 4 illustrates an example of a recommendation interface 400 in accordance with one or more embodiments. The recommendation interface 400 may display information in a table format for easy viewing.

The recommendation interface 400 provides indication of, amongst other fields, an entity category 402, an entity type 404, a model domain display 406, and a domain count 408. The entity category 402 indicates that the category for the recommended entities is a code set. The entity type 404 indicates that the type of entities identified belong to a code set identified as cs_1028: Body Site. The model domain display 406 indicates that the display name for the model domain entity being presented is 101-chest. The domain count 408 indicates the number of comparison domains being considered by the recommendation engine.

The recommendation interface 400 further provides similarity metrics 410, model domain attributes 412, comparison domain attributes 414, ranking categories 416, and domain counts 418 for the entity pairs. The similarity metrics 410 are the similarity values for the entity pairs for the model domain entity identified as 101-chest and the comparison domains. The entity pairs are presented in order of most similar at the top to least similar at the bottom. The model domain attributes 412 are the attributes used by the recommendation engine for creating the vector embedding for the model domain entity. Similarly, the comparison domain attributes 414 are the attributes used by the recommendation engine for creating the vector embeddings for the comparison domain entities. As shown, the model domain attributes 412 and the comparison domain attributes 414 are the same and include “Display,” “Description,” and “Definition.” The ranking categories 416 are the categories that the entity pairs are placed based on the similarity score. Entity pairs with a similarity score above 0.90 are identified as “Likely” and entity pairs with a similarity score below 0.90 are identified as “Possible.” The domain counts 418 indicate the number of comparison domains that the comparison domain entity for the entity pair appears.

### 6. Practical Applications, Advantages & Improvements

In one or more embodiments, standardization of entities of a comparison domain with entities of a model domain facilitates the exchange of data and increases interoperability as data leaving a system will land in the receiving system without loss in fidelity. Standardization of entities reduces variance and cost of maintenance amongst multiple health systems and allows for easy adoption of EHR provider solutions across a client base. Standardization of entities can improve the technical performance of health care systems, for example, by reducing the incidence of data errors when exchanging data between systems.

In one or more embodiments, standardization of entities permits comparing of domains within a client, i.e., production and certification domains. Standardization of entities also allows for comparing data across clients, i.e., when consolidating domains. For example, when consolidating domains, a model domain is the surviving domain, e.g., long-term domain, main domain, master domain, and the comparison domain is the dying domain, e.g., tertiary domain, slave domain, replicated domain.

### 7. Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

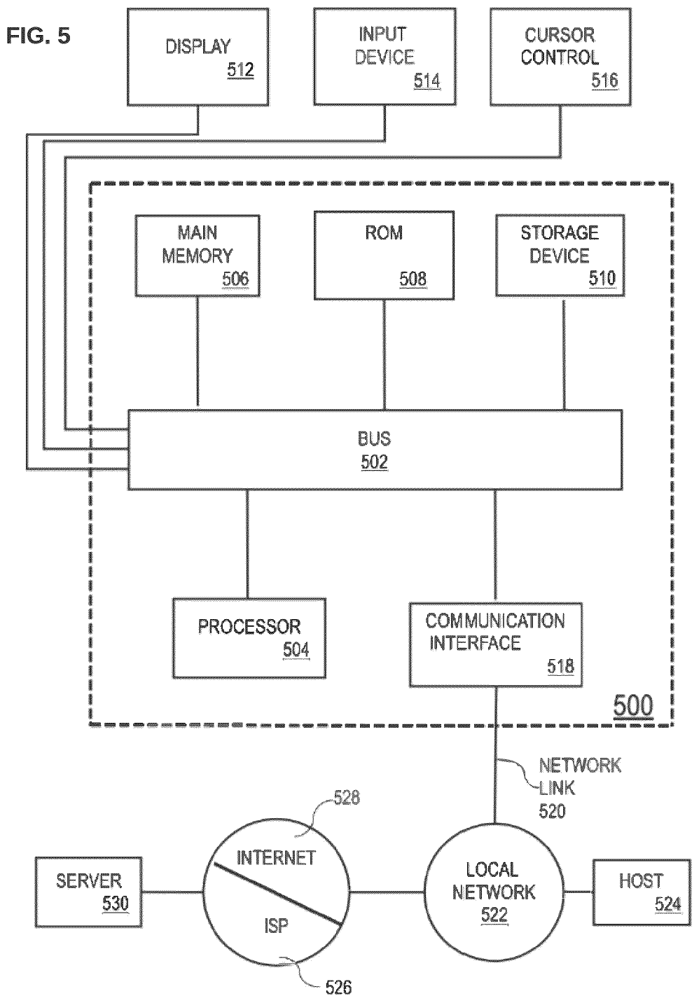

For example, FIG. 5 is a block diagram that illustrates a computer system 500 upon which an embodiment of the disclosure may be implemented. Computer system 500 includes a bus 502 or other communication mechanism for communicating information, and a hardware processor 504 coupled with bus 502 for processing information. Hardware processor 504 may be, for example, a general purpose microprocessor.

Computer system 500 also includes a main memory 506, such as a random access memory (RAM) or other dynamic storage device, coupled to bus 502 for storing information and instructions to be executed by processor 504. Main memory 506 also may be used for storing temporary variables or other intermediate information during execution of instructions to be executed by processor 504. Such instructions, when stored in non-transitory storage media accessible to processor 504, render computer system 500 into a special-purpose machine that is customized to perform the operations specified in the instructions.

Computer system 500 further includes a read only memory (ROM) 508 or other static storage device coupled to bus 502 for storing static information and instructions for processor 504. A storage device 510, such as a magnetic disk, optical disk, or a Solid State Drive (SSD) is provided and coupled to bus 502 for storing information and instructions.

Computer system 500 may be coupled via bus 502 to a display 512, such as a cathode ray tube (CRT), for displaying information to a computer user. An input device 514, including alphanumeric and other keys, is coupled to bus 502 for communicating information and command selections to processor 504. Another type of user input device is cursor control 516, such as a mouse, a trackball, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

Computer system 500 may implement the techniques described herein using customized hard-wired logic, one or more ASICs or FPGAs, firmware and/or program logic which in combination with the computer system causes or programs computer system 500 to be a special-purpose machine. According to one embodiment, the techniques herein are performed by computer system 500 in response to processor 504 executing one or more sequences of one or more instructions contained in main memory 506. Such instructions may be read into main memory 506 from another storage medium, such as storage device 510. Execution of the sequences of instructions contained in main memory 506 causes processor 504 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 510. Volatile media includes dynamic memory, such as main memory 506. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 502. Transmission media can also take the form of acoustic or light waves, such as those generated during radio-wave and infra-red data communications.

Various forms of media may be involved in carrying one or more sequences of one or more instructions to processor 504 for execution. For example, the instructions may initially be carried on a magnetic disk or solid state drive of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector can receive the data carried in the infra-red signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

Computer system 500 also includes a communication interface 518 coupled to bus 502. Communication interface 518 provides a two-way data communication coupling to a network link 520 that is connected to a local network 522. For example, communication interface 518 may be an integrated services digital network (ISDN) card, cable modem, satellite modem, or a modem to provide a data communication connection to a corresponding type of telephone line. As another example, communication interface 518 may be a local area network (LAN) card to provide a data communication connection to a compatible LAN. Wireless links may also be implemented. In any such implementation, communication interface 518 sends and receives electrical, electromagnetic or optical signals that carry digital data streams representing various types of information.

Network link 520 typically provides data communication through one or more networks to other data devices. For example, network link 520 may provide a connection through local network 522 to a host computer 524 or to data equipment operated by an Internet Service Provider (ISP) 526. ISP 526 in turn provides data communication services through the worldwide packet data communication network now commonly referred to as the “Internet” 528. Local network 522 and Internet 528 both use electrical, electromagnetic or optical signals that carry digital data streams. The signals through the various networks and the signals on network link 520 and through communication interface 518, which carry the digital data to and from computer system 500, are example forms of transmission media.

Computer system 500 can send messages and receive data, including program code, through the network(s), network link 520 and communication interface 518. In the Internet example, a server 530 might transmit a requested code for an application program through Internet 528, ISP 526, local network 522 and communication interface 518.

The received code may be executed by processor 504 as it is received, and/or stored in storage device 510, or other non-volatile storage for later execution.

### 8. Miscellaneous; Extensions

Unless otherwise defined, all terms (including technical and scientific terms) are to be given their ordinary and customary meaning to a person of ordinary skill in the art, and are not to be limited to a special or customized meaning unless expressly so defined herein.

This application may include references to certain trademarks. Although the use of trademarks is permissible in patent applications, the proprietary nature of the marks should be respected and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment, one or more non-transitory computer readable storage media comprises instructions which, when executed by one or more hardware processors, cause performance of any of the operations described herein and/or recited in any of the claims.

In an embodiment, a method comprises operations described herein and/or recited in any of the claims, the method being executed by at least one device including a hardware processor.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

What is claimed is:

1. One or more non-transitory computer readable media comprising instructions which, when executed by one or more hardware processors, cause performance of operations comprising:
accessing a first comparison domain entity, from a comparison domain, that describes a first health concept using a first plurality of attributes;
generating a first comparison domain vector embedding for the first comparison domain entity using the first plurality of attributes;
accessing a first model domain entity, from a model domain that describes a second health concept using a second plurality of attributes,
wherein at least a first attribute in the first plurality of attributes differs from at least a second attribute in the second plurality of attributes;
generating a first model domain vector embedding corresponding to the first model domain entity using the second plurality of attributes;
computing a first similarity metric for the first comparison domain vector embedding and the first model domain vector embedding;
based at least on the first similarity metric, presenting the first model domain entity as a candidate model domain entity for mapping to the first comparison domain entity;
receiving user input indicating that the first health concept of the first model domain entity and the second health concept of the first comparison domain entity are a match;
responsive to receiving the user input, updating the model domain to reflect the match between the first health concept of the first model domain entity and the second health concept of the first comparison domain entity; and
exchanging health code data between a first healthcare system and a second healthcare system based on the updated model domain.

2. The non-transitory computer readable media of claim 1, wherein the model domain is associated with an electronic health record (EHR) provider and the comparison domain is associated with a client of the EHR provider.

3. The non-transitory computer readable media of claim 1, wherein the operations further comprise:
determining that the first similarity metric meets or exceeds a threshold value; and
responsive to determining that the first similarity metric meets or exceeds the threshold value: presenting data in a user interface indicating that the first model domain entity and the first comparison domain entity are a likely match for a particular health concept.

4. The non-transitory computer readable media of claim 1, wherein the operations further comprise:
accessing a second model domain entity, from the model domain, that describes a third healthcare concept using a third plurality of attributes;
generating a second model domain vector embedding corresponding to the second model domain entity;
computing a second similarity metric for the first comparison domain vector embedding and the second model domain vector embedding;
based at least on the second similarity metric, refraining from presenting the second model domain entity as any candidate model domain entity for mapping to the first comparison domain entity.

5. The non-transitory computer readable media of claim 1, wherein the operations further comprise:
accessing a second comparison domain entity, from the comparison domain, that describes a third healthcare concept using a third plurality of attributes;
generating a second comparison domain vector embedding corresponding to the second comparison domain entity;
computing a second similarity metric for the second comparison domain vector embedding and the first model domain vector embedding;
based at least on the second similarity metric for the second comparison domain vector embedding and the first model domain vector embeddings, presenting the second comparison domain entity as a candidate entity for adding to the model domain.

6. The one or more non-transitory computer readable media of claim 1, wherein the operations further comprise:
identifying a predetermined number of highest similarity values of a plurality of similarity metrics; and
presenting model domain entities, mapped to vector embeddings that correspond to the predetermined number of highest similarity values, as candidate model domain entities for mapping to the first comparison domain entity.

7. The one or more non-transitory computer readable media of claim 1, wherein the operations further comprise:
accessing a second comparison domain entity, from the comparison domain, that describes a third healthcare concept using a third plurality of attributes;
generating a second comparison domain vector embedding corresponding to the second comparison domain entity;
computing a second similarity metric for the second comparison domain vector embedding and the first model domain vector embedding; and
classifying the first comparison domain entity and the second comparison domain entity into separate categories based at least on the respective first and second similarity scores.

8. A method comprising:
accessing a first comparison domain entity, from a comparison domain, that describes a first health concept using a first plurality of attributes;
generating a first comparison domain vector embedding for the first comparison domain entity using the first plurality of attributes;
accessing a first model domain entity, from a model domain that describes a second health concept using a second plurality of attributes,
wherein at least a first attribute in the first plurality of attributes differs from at least a second attribute in the second plurality of attributes;
generating a first model domain vector embedding corresponding to the first model domain entity using the second plurality of attributes;
computing a first similarity metric for the first comparison domain vector embedding and the first model domain vector embedding;
based at least on the first similarity metric, presenting the first model domain entity as a candidate model domain entity for mapping to the first comparison domain entity;
receiving user input indicating that the first health concept of the first model domain entity and the second health concept of the first comparison domain entity are a match;
responsive to receiving the user input, updating the model domain to reflect the match between the first health concept of the first model domain entity and the second health concept of the first comparison domain entity; and
exchanging health code data between a first healthcare system and a second healthcare system based on the updated model domain,
wherein the method is performed by at least one device including a hardware processor.

9. The method of claim 8, wherein the model domain is associated with an electronic health record (EHR) provider and the comparison domain is associated with a client of the EHR provider.

10. The method of claim 8, further comprising,
determining that the first similarity metric meets or exceeds a threshold value; and
responsive to determining that the first similarity metric meets or exceeds the threshold value: presenting data in a user interface indicating that the first model domain entity and the first comparison domain entity are a likely match for a particular health concept.

11. The method of claim 8, further comprising,
accessing a second model domain entity, from the model domain, that describes a third healthcare concept using a third plurality of attributes;
generating a second model domain vector embedding corresponding to the second model domain entity;
computing a second similarity metric for the first comparison domain vector embedding and the second model domain vector embedding;
based at least on the second similarity metric, refraining from presenting the second model domain entity as any candidate model domain entity for mapping to the first comparison domain entity.

12. The method of claim 8, further comprising,
accessing a second comparison domain entity, from the comparison domain, that describes a third healthcare concept using a third plurality of attributes;
generating a second comparison domain vector embedding corresponding to the second comparison domain entity;
computing a second similarity metric for the second comparison domain vector embedding and the first model domain vector embedding;
based at least on the second similarity metric for the second comparison domain vector embedding and the first model domain vector embeddings, presenting the second comparison domain entity as a candidate entity for adding to the model domain.

13. The method of claim 8, further comprising,
identifying a predetermined number of highest similarity values of a plurality of similarity metrics; and
presenting model domain entities, mapped to vector embeddings that correspond to the predetermined number of highest similarity values, as candidate model domain entities for mapping to the first comparison domain entity.

14. The method of claim 8, wherein the operations further comprise:
accessing a second comparison domain entity, from the comparison domain, that describes a third healthcare concept using a third plurality of attributes;
generating a second comparison domain vector embedding corresponding to the second comparison domain entity;
computing a second similarity metric for the second comparison domain vector embedding and the first model domain vector embedding; and
classifying the first comparison domain entity and the second comparison domain entity into separate categories based at least on the respective first and second similarity scores.

15. A system comprising:
at least one device including a hardware processor;
the system being configured to perform operations comprising:
accessing a first comparison domain entity, from a comparison domain, that describes a first health concept using a first plurality of attributes;
generating a first comparison domain vector embedding for the first comparison domain entity using the first plurality of attributes;
accessing a first model domain entity, from a model domain that describes a second health concept using a second plurality of attributes,
wherein at least a first attribute in the first plurality of attributes differs from at least a second attribute in the second plurality of attributes;
generating a first model domain vector embedding corresponding to the first model domain entity using the second plurality of attributes;
computing a first similarity metric for the first comparison domain vector embedding and the first model domain vector embedding;
based at least on the first similarity metric, presenting the first model domain entity as a candidate model domain entity for mapping to the first comparison domain entity;
receiving user input indicating that the first health concept of the first model domain entity and the second health concept of the first comparison domain entity are a match;
responsive to receiving the user input, updating the model domain to reflect the match between the first health concept of the first model domain entity and the second health concept of the first comparison domain entity; and
exchanging health code data between a first healthcare system and a second healthcare system based on the updated model domain.

16. The system of claim 15, wherein the model domain is associated with an electronic health record (EHR) provider and the comparison domain is associated with a client of the EHR provider.

17. The system of claim 15, wherein the operations further comprise:
determining that the first similarity metric meets or exceeds a threshold value; and
responsive to determining that the first similarity metric meets or exceeds the threshold value: presenting data in a user interface indicating that the first model domain entity and the first comparison domain entity are a likely match for a particular health concept.

18. The system of claim 15, wherein the operations further comprise:
accessing a second model domain entity, from the model domain, that describes a third healthcare concept using a third plurality of attributes;
generating a second model domain vector embedding corresponding to the second model domain entity;
computing a second similarity metric for the first comparison domain vector embedding and the second model domain vector embedding;
based at least on the second similarity metric, refraining from presenting the second model domain entity as any candidate model domain entity for mapping to the first comparison domain entity.

19. The system of claim 15, wherein the operations further comprise:
accessing a second comparison domain entity, from the comparison domain, that describes a third healthcare concept using a third plurality of attributes;
generating a second comparison domain vector embedding corresponding to the second comparison domain entity;
computing a second similarity metric for the second comparison domain vector embedding and the first model domain vector embedding;
based at least on the second similarity metric for the second comparison domain vector embedding and the first model domain vector embeddings, presenting the second comparison domain entity as a candidate entity for adding to the model domain.

20. The system of claim 15, wherein the operations further comprise:
identifying a predetermined number of highest similarity values of a plurality of similarity metrics; and
presenting model domain entities, mapped to vector embeddings that correspond to the predetermined number of highest similarity metrics, as candidate model domain entities for mapping to the first comparison domain entity.
